(19) United States
(12) Patent Application Publication
(10) Pub. No.: US 2024/0274286 A1
(43) Pub. Date: Aug. 15, 2024

(54) Clinical Outcome Prediction By Application Of Machine Learning Models To Clinical Data

(71) Applicant: Bristol-Myers Squibb Company, Princeton, NJ (US)

(72) Inventors: Zekai Chen, Gaithersburg, MD (US); Kevin Alex Brown, Drexel Hill, PA (US)

(73) Assignee: Bristol-Myers Squibb Company, Princeton, NJ (US)

(21) Appl. No.: 18/166,881

(22) Filed: Feb. 9, 2023

(51) Int. Cl. G16H 50/20; G06N 3/0499; G06N 3/08; G16H 10/60

(52) U.S. Cl. CPC G16H 50/20; G06N 3/0499; G06N 3/08; G16H 10/60

(57) ABSTRACT

A method includes receiving a clinical data table for a patient. The clinical data table stores clinical data associated with the patient in tabular form. The method also includes extracting, from the clinical data table, one or more categorical features and one or more continuous features, and determining, using a clinical prediction model, one or more predicted clinical outcomes for the patient based on the one or more categorical features and the one or more continuous features extracted from the clinical data table. The method also includes providing, for output from a client device associated with a user, the one or more predicted clinical outcomes for the patient.

### TECHNICAL FIELD

This disclosure relates to clinical outcome prediction by application of machine learning models to clinical data.

### BACKGROUND

Clinical prediction models play a crucial role in conventional clinical care by predicting outcomes for patients undergoing therapies to treat various medical conditions. In addition to informing professionals, patients, and family members about outcome risks of therapies, the outcomes predicted by clinical prediction models provide an ability to infer accurate treatment responses and disease progress forecasts based on clinical features and a molecular profiles of the patients. In turn, professionals can develop precise medications for eventual use in the medical decision-making process in order to provide personalized treatment decisions for treating a patient's medical condition while considering outcome risks given the patient's clinical features and molecular profile.

### SUMMARY

One aspect of the disclosure provides a computer-implemented method executed on data processing hardware that causes the data processing hardware to perform operations that include receiving a clinical data table for a patient clinical data associated with the patient in tabular form and extracting, from the clinical data table, one or more categorical features and one or more continuous features. The operations also include determining, using a clinical prediction model, one or more predicted clinical outcomes for the patient based on the one or more categorical features and the one or more continuous features extracted from the clinical data table. The operations also include providing, for output from a client device associated with a user, the one or more predicted clinical outcomes for the patient.

Implementations of the disclosure may include one or more of the following optional features. In some implementations, the clinical prediction model executes on the data processing hardware and includes a clinical tabular multi-head attention model. Here, the clinical tabular multi-head attention model includes a categorical feature encoder, a continuous feature encoder, a concatenator, a multi-head attention network, and a fully-connected feed forward network. The categorical feature encoder is configured to receive, as input, each categorical feature of the one or more categorical features extracted from the clinical data table, and generate, as output, a corresponding categorical embeddings for each categorical feature. The continuous feature encoder is configured to receive, as input, each continuous feature of the one or more categorical features extracted from the clinical data table, and generate, as output, a corresponding continuous feature embedding for each categorical feature. The concatenator is configured to concatenate the one or more categorical feature embeddings and the one or more continuous feature embeddings to form a set of parametric embeddings. The multi-head attention network is configured to receive, as input, each parametric embedding in the set of parametric embeddings formed by the concatenator, and generate, as output, a corresponding contextual embedding for each parametric embedding in the set of parametric embeddings. The fully-connected feedforward network is configured to receive, as input, the contextual embeddings generated as output from the multi-head attention network, and predict, as output, the one or more clinical outcomes for the patient. In these implementations, the multi-head attention network includes a stack of N layers that each include a multi-head attention mechanism which may include Transformer layers. Each transformer layer may include a normalization layer, a masked multi-head attention layer, residual connections, and a feedforward layer. Moreover, the one or more clinical outcomes predicted for the patient includes multiple clinical outcomes predicted for the patient and the fully-connected feed forward network includes multiple heads each configured to receive, as input, the contextual embeddings generated as output from the multi-head attention network, and predict, as output, a respective one of the multiple clinical outcomes for the patient. The clinical tabular multi-head attention model is trained via multi-task learning to jointly teach the clinical tabular multi-head attention model to learn how to predict the multiple clinical outcomes for the patient.

In some examples, the clinical prediction model executes on the data processing hardware and includes a large language model. In these examples, the operations may further include serializing the one or more categorical features and the one or more continuous features extracted from the clinical data table into an input text sequence. Here, determining the one or more predicted clinical outcomes for the patient includes processing, using the large language model, the input text sequence to generate the one or more predicted clinical outcomes. The large language model may include a pre-trained large language model and is fine-tuned using few-shot learning. Additionally or alternatively, the large language model may include a domain-specific large language model pre-trained on a vocabulary and/or syntax associated with a particular domain. For instance, the particular domain may include medical terminology.

The one or more predicted clinical outcomes may include at least one of overall survival, progression-free survival, or best overall response. Additionally or alternatively, the one or more predicted clinical outcomes may include at least one of a recommended treatment or a prognostic biomarker score.

Another aspect of the disclosure provides a system that includes data processing hardware and memory hardware in communication with the data processing hardware and storing instructions that cause the data processing hardware to perform operations that include receiving a clinical data table for a patient clinical data associated with the patient in tabular form and extracting, from the clinical data table, one or more categorical features and one or more continuous features. The operations also include determining, using a clinical prediction model, one or more predicted clinical outcomes for the patient based on the one or more categorical features and the one or more continuous features extracted from the clinical data table. The operations also include providing, for output from a client device associated with a user, the one or more predicted clinical outcomes for the patient.

This aspect may include one or more of the following optional features. In some implementations, the clinical prediction model executes on the data processing hardware and includes a clinical tabular multi-head attention model. Here, the clinical tabular multi-head attention model includes a categorical feature encoder, a continuous feature encoder, a concatenator, a multi-head attention network, and a fully-connected feed forward network. The categorical feature encoder is configured to receive, as input, each categorical feature of the one or more categorical features extracted from the clinical data table, and generate, as output, a corresponding categorical embeddings for each categorical feature. The continuous feature encoder is configured to receive, as input, each continuous feature of the one or more categorical features extracted from the clinical data table, and generate, as output, a corresponding continuous feature embedding for each categorical feature. The concatenator is configured to concatenate the one or more categorical feature embeddings and the one or more continuous feature embeddings to form a set of parametric embeddings. The multi-head attention network is configured to receive, as input, each parametric embedding in the set of parametric embeddings formed by the concatenator, and generate, as output, a corresponding contextual embedding for each parametric embedding in the set of parametric embeddings. The fully-connected feedforward network is configured to receive, as input, the contextual embeddings generated as output from the multi-head attention network, and predict, as output, the one or more clinical outcomes for the patient. In these implementations, the multi-head attention network includes a stack of N layers that each include a multi-head attention mechanism which may include Transformer layers. Each transformer layer may include a normalization layer, a masked multi-head attention layer, residual connections, and a feedforward layer. Moreover, the one or more clinical outcomes predicted for the patient includes multiple clinical outcomes predicted for the patient and the fully-connected feed forward network includes multiple heads each configured to receive, as input, the contextual embeddings generated as output from the multi-head attention network, and predict, as output, a respective one of the multiple clinical outcomes for the patient. The clinical tabular multi-head attention model is trained via multi-task learning to jointly teach the clinical tabular multi-head attention model to learn how to predict the multiple clinical outcomes for the patient.

In some examples, the clinical prediction model executes on the data processing hardware and includes a large language model. In these examples, the operations may further include serializing the one or more categorical features and the one or more continuous features extracted from the clinical data table into an input text sequence. Here, determining the one or more predicted clinical outcomes for the patient includes processing, using the large language model, the input text sequence to generate the one or more predicted clinical outcomes. The large language model may include a pre-trained large language model and is fine-tuned using few-shot learning. Additionally or alternatively, the large language model may include a domain-specific large language model pre-trained on a vocabulary and/or syntax associated with a particular domain. For instance, the particular domain may include medical terminology.

The one or more predicted clinical outcomes may include at least one of overall survival, progression-free survival, or best overall response. Additionally or alternatively, the one or more predicted clinical outcomes may include at least one of a recommended treatment or a prognostic biomarker score.

The details of one or more implementations of the disclosure are set forth in the accompanying drawings and the description below. Other aspects, features, and advantages will be apparent from the description and drawings, and from the claims.

### DESCRIPTION OF DRAWINGS

FIG. 1 is a schematic view of an example system that uses a clinical prediction model to predict one or more clinical outcomes from patient clinical data tables.

FIGS. 2A and 2B are schematic views of an example training process for training a clinical tabular transformer (ClinTaT) model to predict clinical outcomes for a patient based on tabular clinical data.

FIG. 3 is a schematic view of an example Transformer layer.

FIG. 4 is a schematic view of an example training process for fine-tuning a pre-trained large language model to predict clinical outcomes for a patient.

FIGS. 5A-5D illustrate example plots depicting performance of the ClinTaT model in predicting clinical outcomes across multiple cancer types.

FIGS. 6A-6C illustrate example plots each depicting performance of the ClinTaT model in predicting the clinical outcomes of overall survival and progression-free survival across the multiple cancer types.

FIG. 7 is a table depicting a comparison of area under the curve (AUC) performance on treatment response prediction between the ClinTaT model and other baselines models.

FIG. 8 is a table depicting a comparison of C-index performance on overall survival prediction between the ClinTaT model and other baselines models.

FIG. 9 is a table depicting a comparison of C-index performance on progression-free survival prediction between the ClinTaT model and other baselines models

FIG. 10 is a table depicting few-shot learning area under the curve (AUC) performance of various large language models pre-trained on different training corpuses.

FIG. 11 is a table evaluating performance changes using different encoder networks stacked on top of various pre-trained large language models for fine-tuning treatment response prediction.

FIG. 12 is a flowchart of an example arrangement of operations for a method of predicting one or more clinical outcomes from tabularized patient data.

FIG. 13 is a schematic view of an example computing device that may be used to implement the systems and methods described herein.

Like reference symbols in the various drawings indicate like elements.

### DETAILED DESCRIPTION

Clinical prediction models play a crucial role in conventional clinical care by predicting outcomes for patients undergoing therapies to treat various medical conditions. In addition to informing professionals, patients, and family members about outcome risks of therapies, the outcomes predicted by clinical prediction models provide an ability to infer accurate treatment responses and disease progress forecasts based on clinical features and molecular profiles of the patients. In turn, professionals can develop precise medications for eventual use in the medical decision-making process in order to provide personalized treatment decisions for treating a patient's medical condition while considering outcome risks given the patient's clinical features and molecular profile.

While machine learning is prevalent for use in training conventional clinical prediction models, the machine learning approaches are typically limited to tree-based ensemble models, such as decision trees, due to the fact that the overwhelming vast majority of the clinical data required for training is stored in tabular form (i.e., clinical data is stored in tables). By contrast to tree-based ensemble models, deep neural networks (DNN) offer many advantages such as the ability to train the resulting model end-to-end, leverage unlabeled/unsupervised training data, are highly robust against both missing and noisy data features, and generally provide better interoperability. DNNs employing the use of multi-head attention mechanisms (e.g., such as Transformers) have revolutionized the fields of natural language processing and computer vision. Yet, these models lack compatibility to train on and interpret data in tabular form.

Implementations herein are directed toward a clinical tabular transformer (ClinTaT) model for predicting clinical outcomes for a patient based on tabular clinical data. Advantageously, the ClinTAT model provides a tabular data modeling architecture using self-attention mechanisms. Examples herein depict Transformers as the type of self-attention mechanisms employed by the ClinTAT model, however, other types of self-attention mechanisms may be employed such as, without limitation, Conformers and lightweight convolution neural networks. The ClinTaT model is particularly effective at modeling continuous features, in addition to categorical features, extracted from patient clinical data tables so that the continuous features are involved in the self-attention modeling process and not dominated by the categorical features during training. This aspect is especially important in the clinical prediction outcome setting where the continuous features represent critical information such as patient age, patient body mass index (BMI), lab results/readouts, and other variables representing the patient's medical profile for use in predicting one or more outcomes related to a patient's medical condition and corresponding therapy the patient is undergoing to treat the patient's medical condition. Implementations further include applying multi-task learning techniques for training the ClinTaT model on a multi-loss objective to teach the ClinTAT model to learn how to predict multiple clinical outcomes for a patient based on clinical data of the patient that is represented by continuous and categorical features extracted from clinical data tables. Example outcomes the ClinTaT model may be trained to learn via multi-task learning may include overall survival (OS) (i.e., in months), progression-free survival (PFS) (in months), and best overall response (BOR). The ClinTaT model may similarly be trained to learn how to predict other outcomes that may include, without limitation, lab results, treatment decisions, disease prediction, drug safety decisions/scores, etc.

Additional implementations are directed leveraging a large language model (LLM) for predicting clinical outcomes for a patient based on an input text sequence serialized from tabular clinical data. Advantageously, LLM's are capable of providing profound in-context learning capabilities when available training samples are limited by exploiting knowledge from other resources to downstream tasks with minimal tuning. By contrast, robustness/accuracy of the ClinTaT model improves when more supervised training samples containing clinical data tables for patients labeled with clinical outcomes is available. Thus, while performance of the ClinTaT model may become degraded when labeled training samples are scarce due to the low inductive bias inherent in long-distance dependency modeling, LLMs are capable of achieving in-context learning capabilities through few-shot learning techniques when only a small number of training samples are available. The use of LLMs may be particularly beneficial for predicting clinical outcomes for rare disease areas where historical patient records are limited, and thus, generally insufficient for training the ClinTAT model. More specifically, these additional implementations are directed toward leveraging LLMs that have been pre-trained on natural language text in the medical domain, and then fine-tuned through few-shot learning by conditioning the domain-specific pre-trained LLMs on available input text sequences serialized from tabular clinical data for predicting specific clinical outcomes. The use of domain-specific LLMs allows for smaller LLMs to be utilized for few-shot learning, thereby reducing processing/memory requirements and training time to fine-tune the LLMs to predict one or more different clinical outcomes through few-shot learning. Example outcomes the LLM model may be trained to learn via a multi-loss objective may include overall survival (OS) (i.e., in months), progression-free survival (PFS) (in months), and best overall response (BOR). The LLM may similarly be trained to learn how to predict other outcomes that may include, without limitation, lab results, treatment decisions, disease prediction, drug safety decisions/scores, etc.

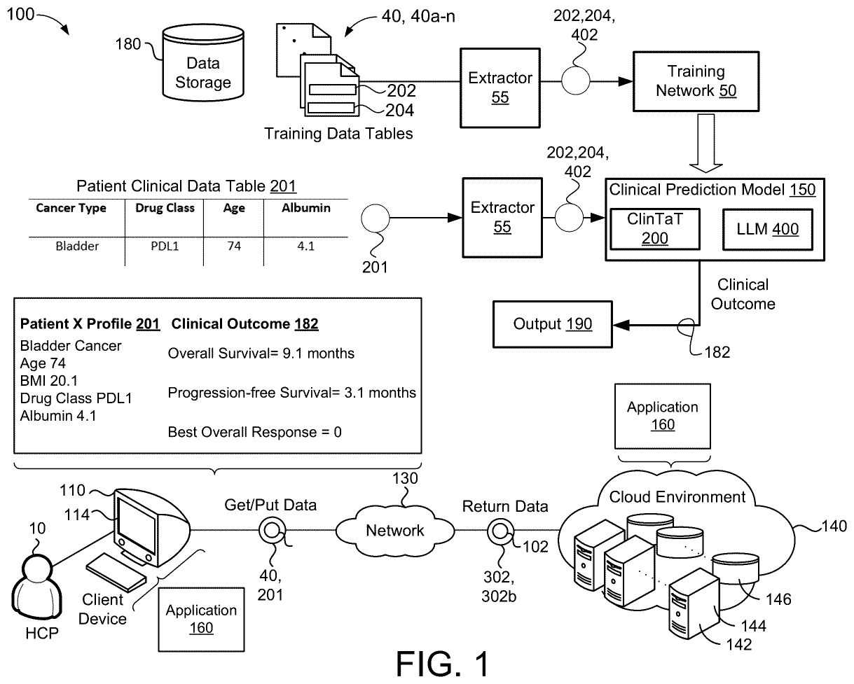

Referring to FIG. 1, in some implementations, a system 100 includes a client device 110 inputting a clinical data table 201 to a clinical prediction model 150 for predicting one or more clinical outcomes 182 for the patient based on the clinical data table 201. The client device 110 is associated with a user 10 such as a healthcare professional (HCP), who may communicate, via a network 130, with a remote system 140. The remote system 140 may be a distributed system (e.g., cloud environment) having scalable/elastic resources 142. The resources 142 include computing resources 144 (e.g., data processing hardware) and/or storage resources 146 (e.g., memory hardware). In some implementations, the remote system 140 executes a clinical prediction application 160 configured to execute the clinical prediction model 150. Here, the client device 110 may access the application 160 running on the remote system 140 and input, via a graphical user interface (GUI) executing on the client device 110, the clinical data table 201 to the clinical prediction model 150. The client device 110 may additionally or alternatively execute the application 160 to implement the ability to run the clinical prediction model 150 on the client device 110 for predicting clinical outcomes 182.

The clinical outcome(s) 182 predicted by the clinical prediction model 150 may inform a patient, healthcare provider, and/or relatives of the patient for making better testing and treatment decisions for a specific health condition is diagnosed with, or for making risk-stratifications for therapeutic trials. For instance, the patient associated with the clinical data table 201 may have metastatic Bladder cancer and is undergoing (or is planned to undergo) immunotherapy to produce antibodies against programmed death-1/programmed death ligand 1 (PD-1/PD-L1) as a form of treatment. In this example, the clinical data table 201 includes various columns of data pertaining to the patient such the type of medical condition (e.g., Bladder cancer) the patient is diagnosed with, immunotherapy drug class (e.g., PDL1) patient characteristics/demographics, laboratory results, imaging tests, and patient history. The patient characteristics/demographics may include the patient's age (e.g., 74), gender, race, ethnicity, height, weight, body mass index (BMI), etc. The laboratory results may include a column for a specific laboratory test and a value indicating a result of the laboratory test. For instance, the column “Albumin” in the data table 201 indicates that the patient's lab result albumin (i.e., level of biomarker albumin) is equal to “4.1”. The clinical data table 201 may include many additional lab results such as hemoglobin and PD-L1 expression measured on a tumor. For instance, PD-L1 expression may form a basis as whether immunotherapy will even be effective since people who have tumors that expressed PD-L1 in 1-percent (1%) or more tumor cells may be more likely to have a durable response than those who have PD-L1 expression that is less than 1-percent (1%). Yet, since there may be other factors where persons with low PD-L1 expressions would still respond remarkably well to immunotherapy, the clinical outcome(s) 182 predicted by the clinical prediction model 150 may help identify whether or not an individual will respond to immunotherapy. The patient history may indicate other details for the patient such as whether or not the patient smokes, consumes alcohol, as well as other diseases/medical conditions the patient has been diagnosed with and/or is being treated for.

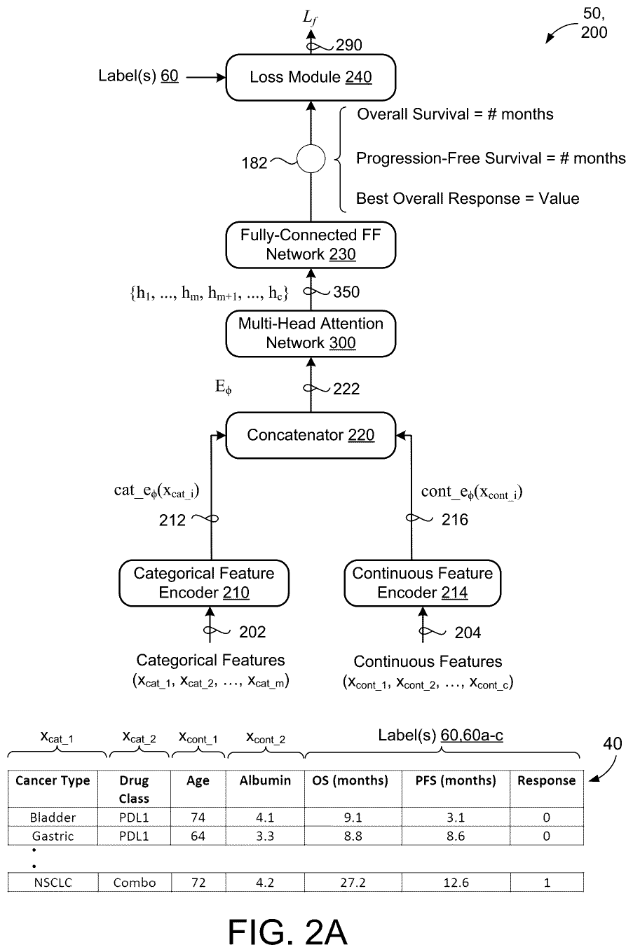

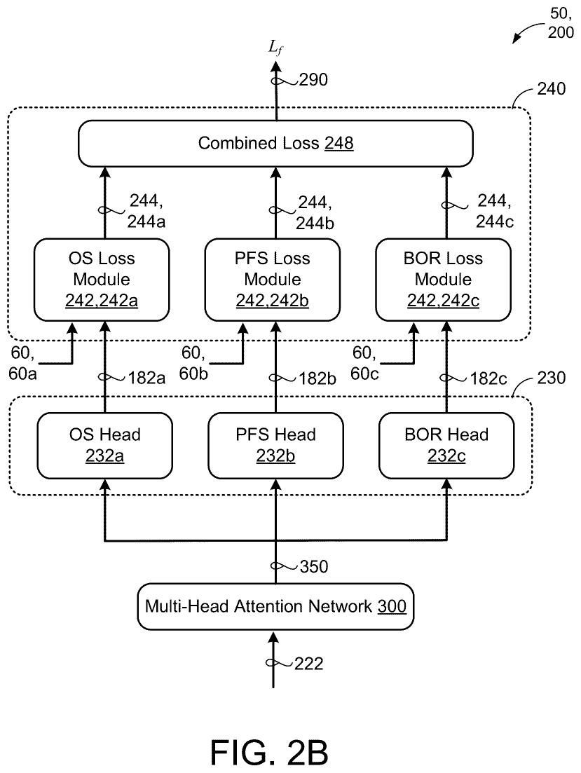

In the example shown, the clinical prediction model 150 corresponds to a cancer prognostic prediction model tasked with predicting clinical outcomes 182 for overall survival (OS), progression-free survival (PFS), and best overall response (BOR). After the clinical prediction model 150 generates/predicts the clinical outcome(s) 182, an output module 190 may provide the clinical outcome(s) 182 for output from the client device 110. In the example shown, the client device 110 receives the clinical outcomes 182 from the clinical prediction model 150 and the GUI executing on the client device 110 displays the clinical outcomes 182 on a screen 114 of the client device 110. The GUI may also present, for display on the screen 114, the categorical and clinical features from the clinical data table 201 associated with the patient. The output module 190 may also store the clinical outcome(s) 182 with the corresponding clinical data table 201 in a data store 180 and/or transmit the clinical outcome 182 to an agency, institution, or other entity. Notably, and as described in greater detail with reference to FIGS. 2A and 2B, the clinical prediction model 150 may be trained using multi-task learning techniques to combine customized loss objectives pertaining to each of the different clinical outcomes 182.

The clinical data table 201 stores the prognostic variables related to the patient's clinical features and molecular profile in tabular form. The values for the various columns may be obtained from various sources and with the consent of the patient. The data stored in the clinical data table 201 may include both categorical features 202 and continuous features 204. The categorical features 202 in the example shown include “Bladder” for the Cancer Type column and PDL1 for the Drug Class column. While not explicitly illustrated in the data table 201, the categorical features 202 may additionally include values that have been scored/binned into categories (e.g., high, low, PDL1 positivity) such as when a given readout value satisfies a threshold value or falls within a range of values. Continuous features 204 on the other hand generally pertain to numerical values such as lab result readouts, patient age, and/or patient BMI/weight. In the example shown, the continuous features 204 include “74” for the Age column and “4.1” for the Albumin column.

In some examples, the user 10 may select/filter the types of clinical data for inclusion in the clinical data table 201 that is being fed to the clinical prediction model 150. Similarly, the user may obtain clinical data tables 201 for a population of patients sharing a particular trait. For instance, the user 10 may use the GUI to provide an input that requests clinical data tables 201 for all patients between the ages of 40-50 and diagnosed with metastatic prostate cancer. In this scenario, the user 10 can obtain clinical outcomes 182 for the patients in the population of interest that exhibit the particular traits (e.g., 40 to 50 years old and diagnosed with prostate cancer). The user 10 may further plug in different values, such as immunotherapy drug class, to see how clinical outcomes 182 predicted for a given patient changes across different immunotherapy drug classes.

In some implementations, the user 10 may be coordinating a clinical trial and may compile clinical data tables 201 for a first group of patients/participants belonging to an active-comparator arm whom are receiving a conventional/effective treatment used in clinical care and also compile clinical data tables 201 for a second group of patients/participants that may join an experimental arm for being treated with a target/experimental therapy. This second group may be a simulated group (or virtual patient population) for testing designs of the clinical trial in a simulated fashion so that patient risks as well as trial costs are substantially reduced. The clinical prediction model 150 may effectively predict the clinical outcomes 182 for these perspective participants in the second group to compare to clinical outcomes 182 for the first group of patients/participants in the active-comparator arm. The comparison results may indicate which candidates are suitable for actually participating in the experimental arm. For instance, it may be revealed that the clinical prediction model 150 predicts clinical outcomes for women under the age of 50 that are not desirable. However, the clinical outcomes 182 predicted for males between the ages of 40-50 may indicate that there is a high likelihood that the target/experimental therapy will be effective for 40 to 50 year old males.

In some implementations, the clinical prediction model 150 includes a clinical tabular transformer (ClinTaT) model 200 that provides a tabular data modeling architecture using self-attention mechanisms. Examples herein depict Transformers as the type of self-attention mechanisms employed by the ClinTAT model 200, however, other types of self-attention mechanisms may be employed such as, without limitation, Conformers and lightweight convolution neural networks. Accordingly, the ClinTaT model may also be referred to as a clinical tabular multi-head attention model. As described in greater detail below with reference to FIGS. 2A and 2B, the ClinTaT model is effective at modeling both the categorical features 202 and continuous features 204 extracted from the clinical data table 201 for a given patient 10.

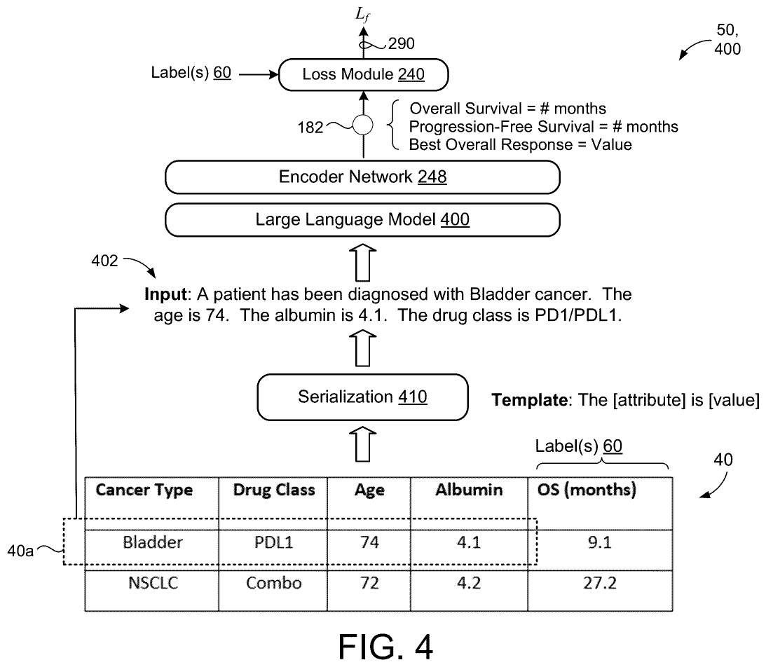

In other implementations, the clinical prediction model 150 includes a large language model (LLM) 400. The LLM 400 provides the ability to provide profound in-context learning capabilities (even when the number of training data tables is sparse) by exploiting knowledge from other resources to downstream tasks with minimal tuning. While the ClinTaT model 200 is suited for tabular data modeling, the LLM 400 is trained to predict clinical outcomes from input text sequences 402 (FIG. 4) serialized from the tabular clinical data 201. Thus, the LLM 400 is configured to process an input text sequence 402 serialized/converted from tabular clinical data 201 to generate the one or more predicted clinical outcomes 182. The LLM 400 may include a domain-specific LLM pre-trained on a vocabulary/syntax associated with the domain such as medical terminology. The use of domain-specific LLMs allows for smaller LLMs to be utilized for few-shot learning, whereby input text sequences 402 serialized from training data tables 40 may be used as context for a query to predict the clinical outcomes 182. Additional implementations are directed leveraging a large language model (LLM) for predicting clinical outcomes for a patient based on an input text sequence serialized from tabular clinical data. Advantageously, LLM's are capable of providing profound in-context learning capabilities when available training samples are limited by exploiting knowledge from other resources to downstream tasks with minimal tuning

A training network 50 is trained on a set of training data tables 40, 40a-n each associated with a respective training patient and including prognostic variables related to the respective training patient's clinical features and molecular profile in tabular form. When the training network 50 is training the ClinTaT model 200, an extractor 55 extracts the categorical features 202 and continuous features 204 from each training data table 40 and provides the extracted categorical and continuous features 202, 204 to the training network 50 for training the ClinTaT model.

Referring now to FIGS. 2A and 2B, the training network 50 trains the ClinTaT model 200 to learn how to predict one or more clinical outcomes 182 from M categorical features (x_{cat_1}, x_{cat_2}, . . . , x_{cat_m}) 202 extracted from each training clinical data table 40 and C continuous features (x_{cont_1}, x_{cont_2}, . . . , x_{cont_c}) 204 extracted from each training clinical data table 40. Each training clinical data table 40 also includes one or more labels 60 that each pertain to a corresponding clinical outcome 182 the model 200 is being trained to learn how to predict. These labels include clinical outcomes actually obtained/recorded for the training patient the corresponding table 40 is associated with. The training patients are completely anonymized. In the example shown, the model 200 is being trained to learn how to predict clinical outcomes 182 including overall survival (OS) (in months), progression-free survival (PFS) (in months), and best overall response (BOR) and the training labels 60 include corresponding columns in the tables 40 for OS (in months), PFS (in months), and BOS (in months). As will become apparent, the labels 60 operate as training targets for a loss module 240 to predict outputs from the ClinTAT model 200 during training. The architecture of the ClinTaT model is particularly effective at modeling continuous features, in addition to categorical features, extracted from patient clinical data tables so that the continuous features are involved in the self-attention modeling process and not dominated by the categorical features during training. This aspect is especially useful in the clinical prediction outcome setting where the continuous features represent critical information such as patient age, patient body mass index (BMI), lab results/readouts, and other variables representing the patient's medical profile for use in predicting one or more outcomes related to a patient's medical condition and corresponding therapy the patient is undergoing to treat the patient's medical condition.

The ClinTaT model 200 includes a categorical feature encoder 210, a continuous feature encoder 214, a concatenator 220, a multi-head attention network 400 (e.g., a stack of N Transformer layers), and a fully-connected feedforward network 230. For each training data table 40, the categorical feature encoder 210 generates, as output, a categorical embedding cat_e_{ϕ}(x_{cat_i}) 212 for a corresponding categorical feature x_{cat_i} 202. In some examples, the categorical feature encoder 210 includes a look-up table of embeddings for the various possible categorical features 202 represented by the columns of the training data table 40. Specifically, for each categorical feature (column) i, the encoder 210 may include an embedding lookup table eφᵢ (.), for i ∈ {1, 2, . . . , m}. For the i-th categorical feature with dᵢ classes, the embedding table eφᵢ (.) has (dᵢ+1) embeddings where the additional embedding corresponds to a missing value. The embedding for the encoded value xᵢ=j ∈ [0, 1, 2, . . . , dᵢ] is e_{φi} (j)=[cφᵢ, wφᵢⱼ], where cφi ∈ R1 and wφij ∈ R_{d-1}. A column-specific and unique identifier cφᵢ ∈ R1 distinguishes the classes in column i from those in the other columns. The dimension of cφᵢ, 1, is a hyper-parameter. Accordingly, the categorical feature encoder 212 outputs a set of categorical embeddings E(x_{cat})={cat_e_{ϕ}(x_{cat_1}), . . . , cat_e_{ϕ}(x_{cat_m})} each representing a corresponding one of the categorical features 202 extracted from the corresponding training data table 40.

For each training data table 40, the continuous feature encoder 214 generates, as output, a continuous embedding cont_e_{ϕ}(x_{cont_i}) 216 for a corresponding continuous feature x_{cont_i} 204. While the categorical feature encoder 210 includes a dictionary-style look-up table of embeddings where a disparate number of a token representing the categorical feature corresponds to an embedding, the continuous feature encoder 214 applies a linear neural network layer to multiply the number representing the corresponding categorical feature 204 (e.g., “74” for the age column or “4.1” for the Albumin lab result column) to obtain a respective sequence of embeddings corresponding to the categorical feature 204. By doing so, the continuous embeddings 216 can be involved in the self-attention modeling process so that the corresponding continuous features 204 are not dominated by the categorical features 202 during training. Notably, the continuous embedding 216 generated for each corresponding continuous feature 204 may also include a column-specific and unique identifier cφᵢ ∈ R1 that distinguishes the value of the continuous feature 204 represented in the column i from the values represented in the other columns in the training data table 40. Accordingly, the continuous feature encoder 214 outputs a set of continuous embeddings E(x_{cont})={cont_e_{ϕ}(x_{cont_1}), . . . , cont_e_{ϕ}(x_{cont_c})} each representing a corresponding one of the continuous features 204.

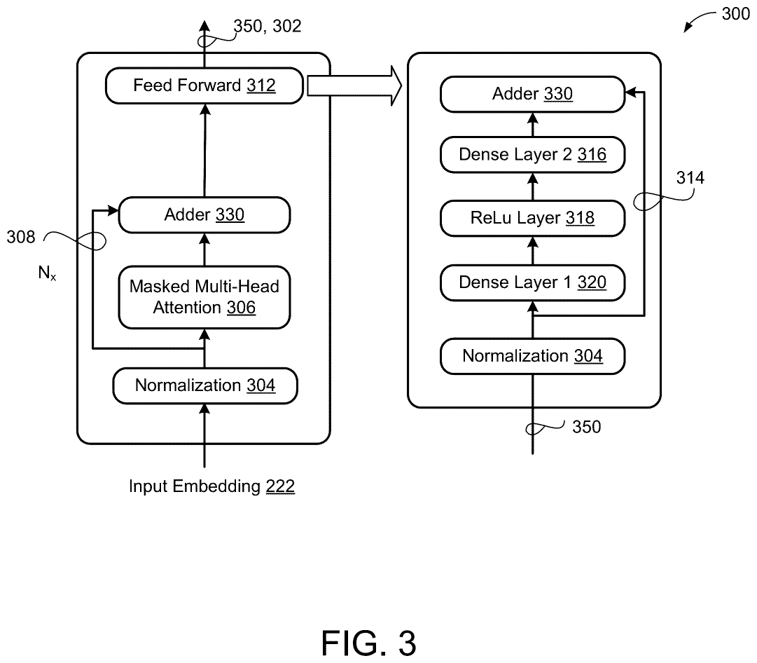

The concatenator 220 concatenates the set of categorical embeddings E(x_{cat}) and the set of continuous embeddings E(x_{cont}) to form a set of parametric embeddings E_{ϕ} 222, wherein E_{ϕ}={cat_e_{ϕ}(x_{cat_1}), . . . , cat_e_{ϕ}(x_{cat_m})}+{cont_e_{ϕ}(x_{cont_1}), . . . , cont_e_{ϕ}(x_{cont_c})}. The multi-head attention network 300 may include a stack of N layers each including a respective multi-head attention mechanism (FIG. 3) 306. The multi-head attention network 300 receives each corresponding parametric embedding from the set of parametric embeddings E_{ϕ} 222 that includes either a categorical embedding cat_e_{ϕ}(x_{cat_i}) 212, output from the categorical feature encoder 210 for a corresponding categorical feature x_{cat_i}, or a continuous embedding cont_e_{ϕ}(x_{cont_i}) 216, output from the continuous feature encoder 214 for a corresponding categorical feature x_{cont_i} 204. The multi-head attention network 300 is configured to generate/transform, for each categorical embedding cat_e_{ϕ}(x_{cat_i}) 212 and each continuous embedding cont_e_{ϕ}(x_{cont_i}) 216, a corresponding contextual embedding 350 through successive aggregation of context from other embeddings 212, 216 in the set of parametric embeddings E_{ϕ}. The multi-head attention network 300 represented by the stack of N layers (e.g., a stack of N Transformer layers) may be denoted as having a function f_{θ} that operates on the set of parametric embeddings E_{ϕ} and returns the corresponding contextual embeddings 350 as {h₁, . . . , hₘ, hₘ₊₁, . . . , h_{c}}, wherein hᵢ hᵢ ∈ R^{d} for i ∈ {1, . . . , m, m+1, . . . c}. Notably, m+1 denotes the first continuous feature (x_{cont_1}) 204 in the sequence continuous features input to the continuous feature encoder 214. As depicted in FIG. 2A, the contextual embeddings 350 {h₁, . . . , hₘ, hₘ₊₁, . . . , h_{c}} form a context vector of dimension (dx(m+c)) that is provided as input to the fully-connected feedforward network 230, which may include multilayer perceptron (MLP).

FIG. 3 shows an example transformer layer 300 among a plurality of transformer layers when the multi-head attention network 300 includes the plurality of transformer layers. As aforementioned, the multi-head attention network 300 of FIGS. 2A and 2B is not limited to transformer layers and may instead include conformer layers, lightweight convolution layers, or other networks employing multi-head attention mechanisms. In FIG. 3, an initial transformer layer 300 receives a corresponding parametric embedding 222 from the set of parametric embeddings E_{ϕ}(x_{cat}+x_{cont}) that includes either a categorical embedding cat_e_{ϕ}(x_{cat_i}) 212, output from the categorical feature encoder 210 for a corresponding categorical feature x_{cat_i}, or a continuous embedding cont_e_{ϕ}(x_{cont_i}) 216, output from the continuous feature encoder 214 for a corresponding categorical feature x_{cont_i} 204, and generates a corresponding output representation/embedding 350 received as input by the next transformer layer 300. That is, each transformer layer 300 subsequent the initial transformer layer 300 may receive an input embedding 350 that corresponds to the output representation/embedding generated as output by the immediately preceding transformer layer 300. The final transformer layer 300 (e.g., the last transformer layer in the stack of transformer layers 300) generates/transforms, for each categorical embedding cat_e_{ϕ}(x_{cat_i}) 212 and each continuous embedding cont_e_{ϕ}(x_{cont_i}) 216, a corresponding contextual embedding 350 through successive aggregation of context from other embeddings 212, 216 in the set of parametric embeddings E_{ϕ}, wherein E_{ϕ}={cat_e_{ϕ}(x_{cat_1}), . . . , cat_e_{ϕ}(x_{cat_m}), cont_e_{ϕ}(x_{cont_1}), . . . , cont_e_{ϕ}(x_{cont_c})}.

Each transformer layer 300 of the multi-head attention network includes a normalization layer 304, masked multi-head attention layer 306, residual connections 308, and a feedforward layer 312. The masked multi-head attention layer 306 provides a flexible way to control the amount of context that the model 200 uses. Specifically, after the normalization layer 304 normalizes the input parametric embedding 222, the masked multi-head attention layer 306 projects the input to a value for all the heads. Thereafter, the masked multi-head layer 306 may mask an attention score to the current parametric embedding 222 to produce an output conditioned on the set of parametric embeddings E_{ϕ}. Then, weight-averaged values for all the heads are concatenated and passed to a dense layer 2 316, where a residual connection 314 is added to the normalized input and the output of the dense layer 316 to form the final output of the multi-head attention layer 306. The residual connections 308 are added to the output of the normalization layer 304, by an adder 330, and are provided as inputs to a respective one of the masked multi-head attention layer 306 or the feedforward layer 312.

The feedforward layer 312 applies normalization layer 304, followed by dense layer 1 320, rectified linear layer (ReLu) 318, and dense layer 2 316. The ReLu 318 is used as the activation on the output of dense layer 1 320. Like in the multi-head attention layer 406, a residual connection 314 of the output from the normalized layer 404 may be added to the output of the dense layer 2 316 by the adder 330.

Referring back to FIG. 2A, based on the set of contextual embeddings 350 for each corresponding training data table 40, the fully-connected feedforward network 230 generates, as output, one or more predicted clinical outcomes 182 and the loss module 240 generates a training loss 290 based on the predicted clinical outcome(s) 182 and the corresponding training label(s) 60 for the training data table. Here, the training labels 60 may include corresponding columns in the table 40 for OS (in months), PFS (in months), and BOS (in months) and operate as training targets for the training network 50 to teach the ClinTaT model 200 to predict the clinical outcomes 182 from the tabularized data 202, 204. Accordingly, the training network 50 trains the ClinTaT model 200 via supervised learning by updating parameters of the ClinTaT model 200 based on the training loss 290 obtained for each training data table 40. For instance, parameters/weights of the categorical feature encoder 210, the continuous feature encoder 214, the multi-head attention network 300, and the fully-connected feedforward network 230.

FIG. 2B shows how the training network 50 applies multi-task learning techniques for training the ClinTaT model 200 on an multi-loss objective to teach the ClinTAT model 200 to learn how to predict multiple clinical outcomes 182, 182a-c. Continuing with the example, the multiple clinical outcomes 182 the ClinTaT model may be trained to learn via multi-task learning may include overall survival (OS) (i.e., in months), progression-free survival (PFS) (in months), and best overall response (BOR). The ClinTaT model may similarly be trained to learn how to predict other outcomes that may include, without limitation, lab results, treatment decisions, etc.

In the example shown, the fully-connected feedforward network 230 includes multiple heads 232a-c each trained to output/generate a respective one of the multiple clinical outcomes 182 based on the contextual embeddings 350 {h₁, . . . , hₘ, hₘ₊₁, . . . , h_{c}} generated by the multi-head attention network 300 for the corresponding categorical and continuous features 202, 204 of each training data table 40. For instance, the OS head 232a is configured to output a predicted overall survival 182a as a value representing a number of months, the PFS head 232b is configured to output a predicted progression-free survival as a value representing a number of months, and the BOR head 232c is configured to output value for a predicted best overall response. Each head 232a-c may include one single linear projection layer that corresponds to the respective clinical outcome 182 the head is predicting.

The loss module 240 includes a plurality of sub-loss modules 242a-c each associated with a respective one of the heads 232a-c. Each sub-loss module 242 is configured to determine a respective sub-loss 244, 244a-c based on the corresponding clinical outcome 182 predicted by the respective head 232a-c and the training label 60a-c that pertains to the corresponding clinical outcome 182. For instance, the sub-loss module 242a may correspond to an OS loss module that determines an OS loss 244a based on the predicted overall survival 182a output from the OS head 232a and the training label 60a that pertains to the ground-truth value the OS head 232a is learning to predict. In some examples, the OS loss 244a includes a Cox Proportional Hazard (CPH) loss function. The sub-loss module 242b may correspond to a PFS loss module that determines a PFS loss 244b based on the predicted PFS 182b output from the PFS head 232b and the training label 60b that pertains to the ground-truth value the PFS head 232b is learning to predict. In some examples, the PFS loss 244b includes a Cox Proportional Hazard (CPH) loss function. The sub-loss module 242c may correspond to a BOR loss module that determines a BOR loss 244c based on the predicted BOR 182c output from the BOR head 232c and the training label 60c that pertains to the ground-truth value the BOR head 232c is learning to predict. In some examples, the BOR loss 244c includes a CrossEntropy loss function. Thus, the sub-losses 244 determined by the sub-loss modules 242 provide a multi-loss objective for enabling the ClinTaT model 200 to predict multiple endpoints (i.e., clinical outcomes 182), and in turn, introduces an inductive bias to allow the model 200 to prefer some predictions over others to lead to better generalization.

With continued reference to FIG. 2B, the loss module 240 includes a combined loss module 248 to provide the joint learning paradigm by summing the different sub-losses 244 each pertaining to a respective one of the different clinical outcomes 182 predicted by the fully-connected feedforward network 230 into a total loss 290 represented by a unified loss objective L_{f}. The L_{f} may be represented as follows.

$${L}_{f} = {\sum }_{i}^{I} {α}_{i} {l}_{i} \qquad ( 1 )$$

where I denotes a total number of tasks and αᵢ denotes a soft weight for any task i.

After the ClinTaT model 200 is trained, the trained ClinTaT model 200 may be used as the clinical prediction model 150 for predicting the one or more clinical outcomes 182 from the clinical data table 201 of a corresponding patient. Referring back to FIG. 1 with reference to FIGS. 2A and 2B, the extractor 55 may extract all the categorical features 202 and the continuous features 204 from the clinical data table 201. Thereafter, the categorical feature encoder 210 may produce categorical embeddings 212 from the categorical features 202 while the continuous feature encoder 214 may produce continuous embeddings 216 from the continuous features 204. The concatenator 220 may then concatenate the categorical and continuous embeddings 212, 216 to form a corresponding set of parametric embeddings E_{ϕ} 222 that are input to the multi-head attention network 300. The multi-head attention network 300 may apply the function f_{θ} to operate on the set of parametric embeddings E_{ϕ} and return corresponding contextual embeddings 350 input to the fully-connected feedforward network 230. Based on the contextual embeddings 350, the feedforward network 230 may predict, as output, the one or more clinical outcomes 182. Continuing with the example, the feedforward network 230 may include an OS head 232a that outputs a predicted OS 182a based on the clinical data table 201, a PFS head 232b that outputs a predicted PFS 182b based on the clinical data table 201, and a BOR head 232c that outputs the predicted BOR 182c. As shown in FIG. 1, the output module 190 may present the predicted OS 182a as “Overall Survival—9.1 months”, the predicted PFS 182b as “Progression-free Survival—3.1 months”, and the predicted BOR 182c as “Best Overall Response—0” for display on the screen 114 of the client device 110.

FIG. 4 shows the training network 50 training the LLM 400 on the training data tables 40 to teach the LLM to learn how predict the one or more clinical outcomes 182. As mentioned previously, the training network 50 may train the LLM 400 for use as the clinical prediction model 150 when available training data (e.g., training data tables) is sparse, or not sufficient for training the ClinTaT model 200, since LLMs are capable of providing profound in-context learning capabilities when available training samples are limited by exploiting knowledge from other resources to downstream tasks with minimal tuning.

The training network 50 applies serialization 410 to serialize the tabular clinical data stored in each training data table 40 into a corresponding input text sequence 402. Here, the input text sequence 402 serialized from the features in the column of each training data table 40 includes a sequence of natural language tokens (e.g., words/wordpieces) that the LLM is able to comprehend and encode. In some examples, the serialization 410 applies a manual serialization template to the feature of each column. For instance, the manual serialization template may include “The {attribute} is {value}.” In the example shown, for a first training data table 40a having values in columns representing attributes of Cancer Type, Drug Class, Age, and Albumin, the manual serialization template applied by the serialization 410 produces the input text sequence 402 “The patient has been diagnosed with Bladder Cancer. The age is 74. The albumin is 4.1. The drug class is PD1/PDL1.” Other attributes such any prognostic or predictive biomarker can also be used in the methods and systems described herein.

The input text sequence 402 serialized from each training data table 40 is provided as input to the LLM 400 for predicting one or more clinical outcomes 182 therefrom. Implementations herein are directed toward the LLM 400 including a pre-trained LLM and the training network 50 performing few-shot learning using the input text sequence 402 serialized from each training data table 40 as context for predicting the one or more clinical outcomes 192. The pre-trained LLM 400 may include a domain-specific LLM pre-trained on a vocabulary/syntax associated with the domain such as medical terminology. The use of domain-specific LLMs allows for smaller LLMs to be utilized for few-shot learning to fine-tune the LLM to predict clinical outcomes, whereby input text sequences 402 serialized from training data tables 40 may be used as context for a query to predict the clinical outcomes 182. In some examples, the training labels 60 are serialized into a corresponding natural language query provided to the LLM 400 to predict the clinical outcomes such that the input text sequences 402 serialized from the training data tables 40 may be used as the context for the natural language query to predict the clinical outcomes. Additionally, using few-shot learning to fine tune the LLM allows for smaller LLMs that require reduced processing/memory requirements and improved latency for making robust and accurate clinical predictions compared to domain-agnostic LLMs that contain billions of more parameters.

The pre-trained LLM may include a Bidirectional Encoder Representation from Transformers (BERT) model or a domain-specific LLMs pre-trained on clinical and/or biomedical corpora. Example domain-specific LLMs may include, without limitation, BioBERT, ClinicalBERT, SciBERT, PubMedBERT.

With continued reference to FIG. 4, the training network 50 may also apply an encoder network 248 stacked on the LLM 400 for fine-tuning the LLM 400 on the multi-loss objective to teach the LLM 400 to predict the clinical outcomes 182, 182a-c. In some examples, the encoder network 248 includes a single linear layer or a stack of multi-head attention layers. For instance, the multi-head attention layers could include Transformer layers or Conformer layers. In some examples, the LLM 400 receives the one or more clinical outcomes for the LLM 400 to predict as a query that requests. The encoder network 248 may receive embeddings output from the LLM 400 and predict, as output, the one or more clinical outcomes 182 and the loss module 240 generates the training loss 290 based on the predicted clinical outcome(s) 182 and the corresponding training label(s) 60 for the training data table. Here, the training labels 60 may include corresponding columns in the table 40 for OS (in months), PFS (in months), and BOS (in months) and operate as training targets for the training network 50 to fine-tune the LLM 400 and the encoder network 248 to predict the clinical outcomes 182 from the input text sequence 402. During fine-tuning, the parameters of the pre-trained LLM 400 may be held fixed/frozen while parameters of the encoder network 248 are tuned/updated based on the training loss 290.

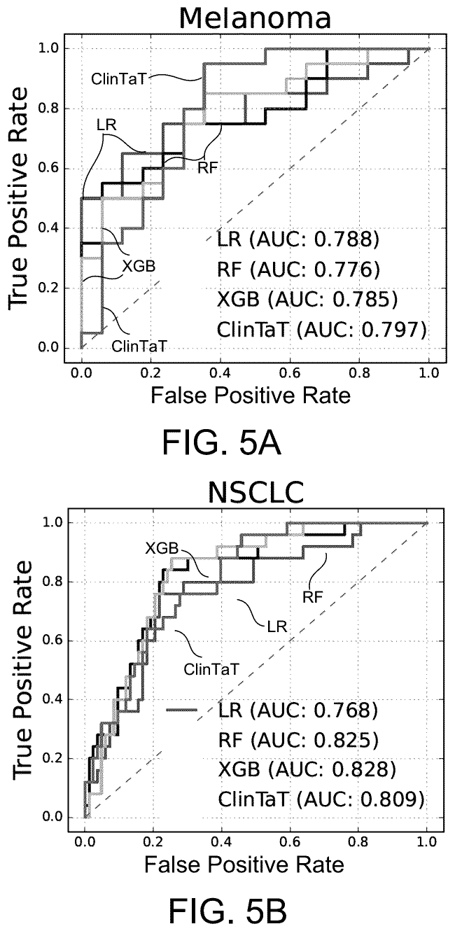

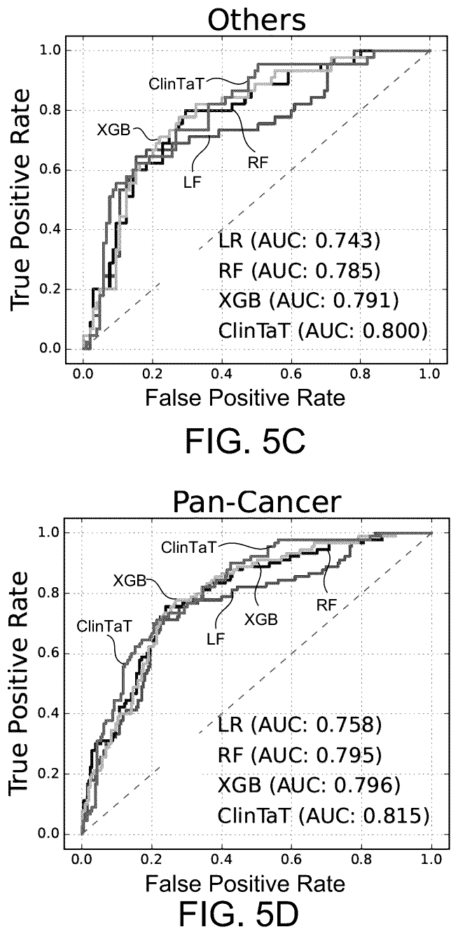

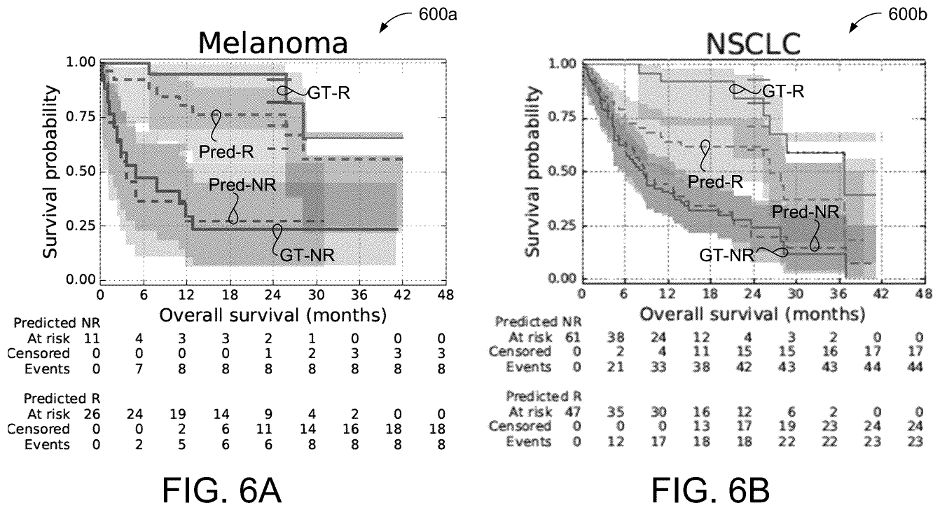

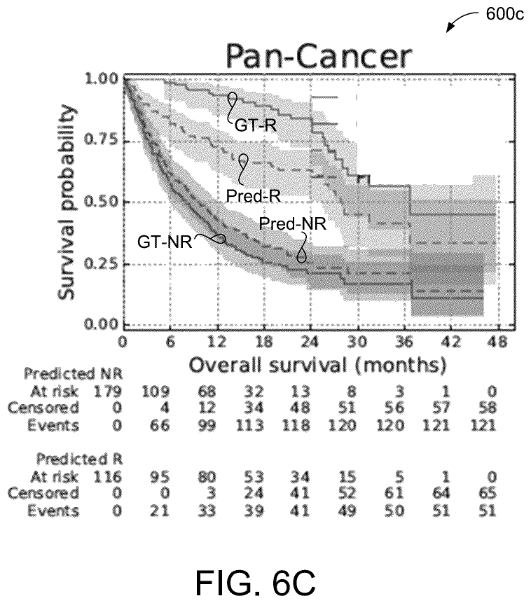

FIGS. 5A-6C illustrate example plots depicting performance of the ClinTAT model 200 in predicting clinical outcomes. In these examples, the ClinTaT model 200 was trained on training data samples acquired by Memorial Sloan Kettering Cancer Center (MSKCC) from a comprehensively curated cohort (MSK-IMPACT) with 1,479 patients treated with immune check-point blockade (ICB) across 16 different cancer types, where patients are either responders (R) or non-responders (NR) to the treatment (PD-1/PD-L1 inhibitors, CTLA-4 blockade or a combination) based on Response Evaluation Criteria in Solid Tumors (RECIST) v1.1 or best overall response on imaging. Each patient was collected up to 16 biological features, including genomic, molecular, clinical, and demographic variables (i.e., represented as corresponding categorical or continuous features 202, 204). The training set contains training data tables 40 for 1,184 patients, and the test set contains clinical data tables for 295 patients. The evaluation target is to predict clinical outcome to immunotherapy (binary classification) and both overall survival and progression-free survival (regression) in the test data across different cancer types.

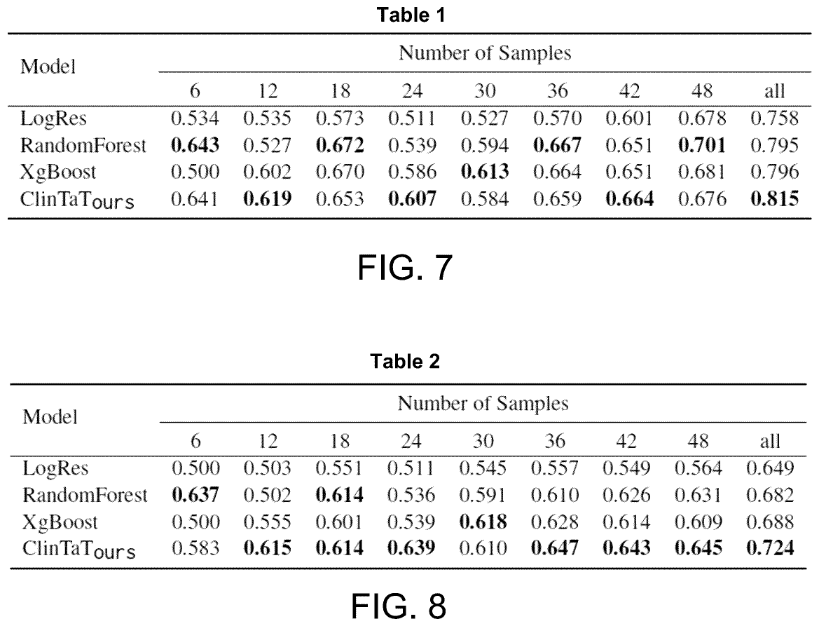

FIGS. 5A-5D illustrate example plots 500a-d each depicting performance of the ClinTaT model 200 in predicting the clinical outcomes across multiple cancer types. The y-axis of each plot 500a-d denotes a true positive rate while the x-axis of each plot denotes a false positive rate. Each plot provides a comparison of predictive performance on the MSK-IMPACT in terms of receiver operating characteristic (ROC) curves and area under the curve (AUC) between ClinTAT and other baselines in each of melanoma (plot 500a of FIG. 5A), non-small cell lung cancer (NSCLC) (plot 500b of FIG. 5B), other cancer types (plot 500c of FIG. 5C), and Pancreatic cancer (plot 500d of FIG. 5D). The ROC curves were calculated using response probabilities computed by transformers and the other baselines. The other baselines include a logistic regression (LR), random forest (RF), and XgBoost models. The ClinTAT achieved superior performance on the test set, as indicated by the AUC in each of the plots 500a-d, in predicting responders and non-responders across cancer types compared to conventional machine learning models such as LR, RF, and XgBoost. The results suggest that the self-attention mechanism for long-range dependency modeling contributed to the overall prediction performance to various degrees. Table 1 of FIG. 7 depicts AUC performance on treatment response prediction of the ClinTaT and the other baselines on the MSK IMPACT, wherein each column reports the k-shot performance for different values of k. Notably, ClinTaT outperforms the other baselines when all training samples are used, however less significant when less training samples are used.

FIGS. 6A-6C illustrate example plots 600a-c each depicting performance of the ClinTaT in predicting the clinical outcomes of OS and PFS across the multiple cancer types on the test data. The y-axis of each plot 600a-c denotes survival probability and the x-axis of each plot 600a-c denotes overall survival (months). The plots 600a-c each plot comparisons between ground-truth responders (GT-R), predicted responders (Pred-R), ground-truth non-responders (GT-NR), and predicted non-responders (Pred-NR). The plots 600a-c reveal that the differences in overall survival between responders and non-responders predicted by transformers across the various cancer types of melanoma (plot 600a of FIG. 6A), non-small cell lung cancer (NSCLC) (plot 600b of FIG. 6B), and pancreatic cancer (plot 600c of FIG. 6C). Especially for the predicted non-responders, the predicted survival curves almost fit the ground-truth ones perfectly, indicating that transformers tend to underestimate the response probability to some extent.

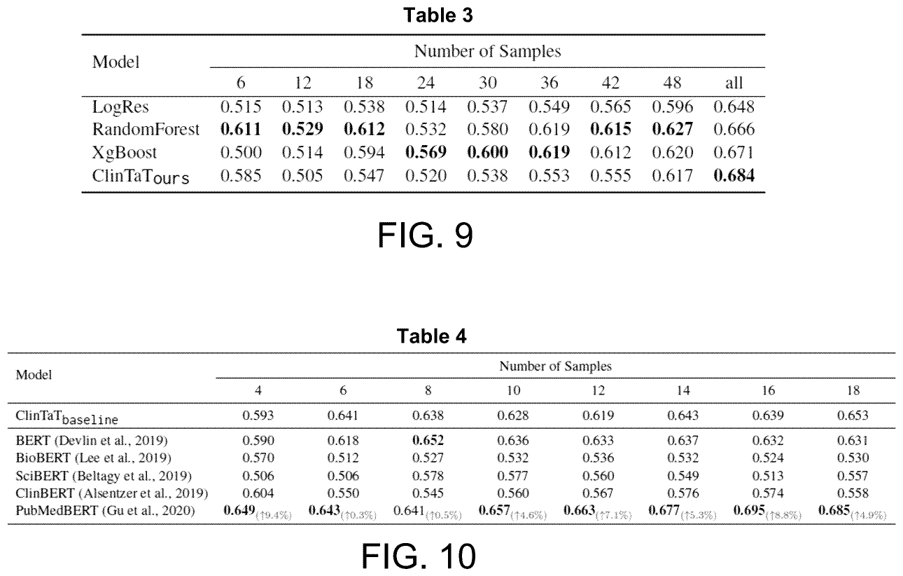

To test whether the ClinTaT model 200 could also predict overall survival (OS) before the administration of immunotherapy, a concordance index (C-index) was calculated for OS and PFS, which ranges between 0 and 1 (0.5 being random performance). Table 2 of FIG. 8 and Table 3 of FIG. 9 indicate that the C-indices of the ClinTaT predictions were significantly higher than those generated by other baselines (Table 2, pan-cancer C-index 0.724 for ClinTaT versus 0.688 for Xg-Boost versus 0.682 for Random Forest, p<0.05; Table 3, pan-cancer C-index 0.684 for ClinTaT versus 0.671 for XgBoost versus 0.666 for Random Forest, p<0.05). These results demonstrate that the transformers can accurately forecast response, OS, and PFS before administering immunotherapy.

Table 4 of FIG. 10 shows the performance of different BERT LLMs pre-trained on different resource corpus followed by a single linear layer as the encoding network 248 (FIG. 4) for fine-tuning using only a [cls] token on the MSK IMPACT test data (averaged over three seeds). The PubMedBERT outperforms all other variants and the base-line transformer across all k-shot settings with an average of improvements over 5%. In the very few shot settings (4 samples), the language model fine-tuning shows significant improvements over the baseline (Table 4, 9.4%), indicating the benefit of the capability of knowledge transferring to down-stream tasks brought by LLMs when samples are insufficient. Also, the results indicate that the sample efficiency of using embeddings output from the LLM is highly domain knowledge dependent. For instance, the performance of SciBERT is worse than that of BioBERT and ClinicalBERT as SciBERT was pre-trained on all semantic scholar 1.14 M articles towards a more general scientific knowledge learning.

In contrast, BioBERT and ClinicalBERT were pre-trained on the more domain-specific corpus, such as PubMed, PMC, and clinical MIMIC III notes (available at mimic.mit.edu). A preliminary conjectures is that domain-specific knowledge transfer may be superior when the pre-training corpus is sufficiently profound. However, the generalization capability learned by domain-agnostic models also works under scenarios where the resource knowledge is neither domain-agnostic nor morally domain-specific.

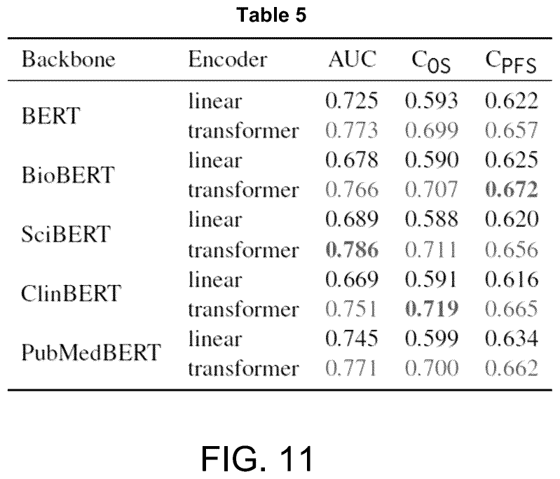

Though all the results in Table 4 are generated by adding one single linear layer on top of LLMs for fine-tuning, Table 5 of FIG. 11 evaluates the performance change using different encoder networks 238 (FIG. 4). The transformer in Table 5 includes only a transformer encoder of a depth of six layers with a dimension of 768. The results indicate that adding compute complexity to LLMs can still lift the semantic representation learning of clinical features, as transformer architecture performs better than a superficial linear layer.

A software application (i.e., a software resource) may refer to computer software that causes a computing device to perform a task. In some examples, a software application may be referred to as an “application,” an “app,” or a “program.” Example applications include, but are not limited to, system diagnostic applications, system management applications, system maintenance applications, word processing applications, spreadsheet applications, messaging applications, media streaming applications, social networking applications, and gaming applications.

The non-transitory memory may be physical devices used to store programs (e.g., sequences of instructions) or data (e.g., program state information) on a temporary or permanent basis for use by a computing device. The non-transitory memory may be volatile and/or non-volatile addressable semiconductor memory. Examples of non-volatile memory include, but are not limited to, flash memory and read-only memory (ROM)/programmable read-only memory (PROM)/erasable programmable read-only memory (EPROM)/electronically erasable programmable read-only memory (EEPROM) (e.g., typically used for firmware, such as boot programs). Examples of volatile memory include, but are not limited to, random access memory (RAM), dynamic random access memory (DRAM), static random access memory (SRAM), phase change memory (PCM) as well as disks or tapes.

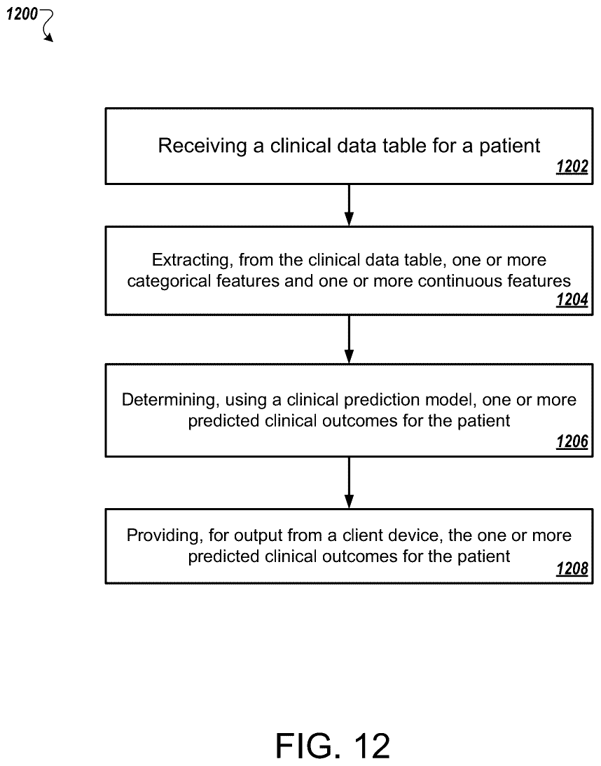

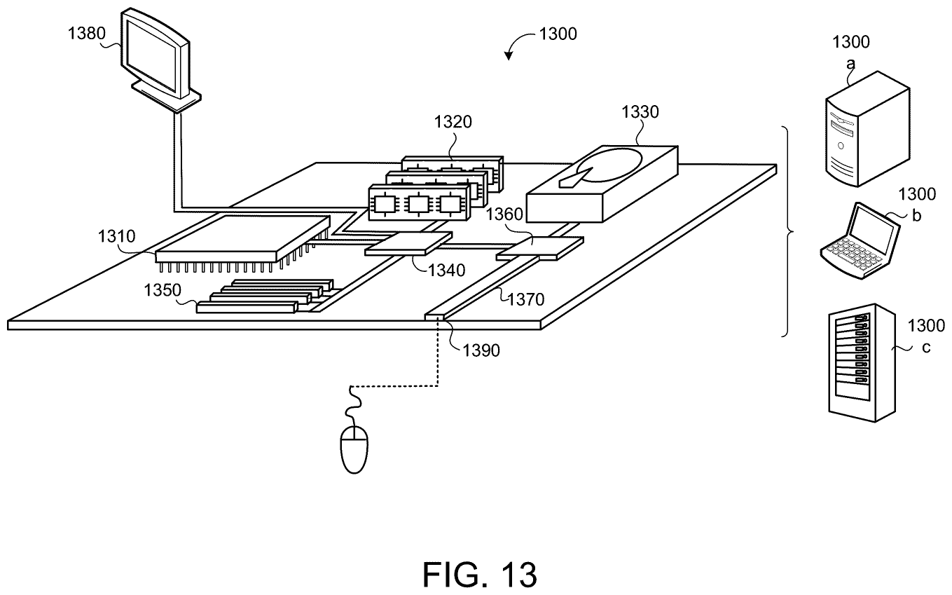

FIG. 12 provides a flowchart of an example arrangement of operations for a method 1200 of predicting clinical outcomes from patient data stored in tabularized form. The method 1200 may execute on data processing hardware 1310 (FIG. 13) based on instructions stored on memory hardware 1320 (FIG. 13) that cause the data processing hardware 310 to perform the operations. The data processing hardware 1310 and the memory hardware 1320 may include the data processing hardware 144 and the memory hardware 146 of the remote system 140. Additionally or alternatively, the data processing hardware 130 and the memory hardware 1320 may reside on the client device 110.

At operation 1202, the method 1200 includes receiving a clinical data table 201 for a patient. Here, the clinical data table 201 stores clinical data associated with the patient in tabular form. At operation 1204, the method 1200 includes extracting, from the clinical data table 201, one or more categorical features 202 and one or more continuous features 204.

At operation 1206, the method 1200 includes determining, using a clinical prediction model 150, one or more predicted clinical outcomes 182 for the patient based on the one or more categorical features 202 and the one or more continuous features 204 extracted from the clinical data table 201. At operation 1208, the method 1200 includes providing, for output from a client device 110 associated with a user 10, the one or more predicted clinical outcomes 182 for the patient.

FIG. 13 is schematic view of an example computing device 1300 that may be used to implement the systems and methods described in this document. The computing device 1300 is intended to represent various forms of digital computers, such as laptops, desktops, workstations, personal digital assistants, servers, blade servers, mainframes, and other appropriate computers. The components shown here, their connections and relationships, and their functions, are meant to be exemplary only, and are not meant to limit implementations of the inventions described and/or claimed in this document.

The computing device 1300 includes a processor 1310, memory 1320, a storage device 1330, a high-speed interface/controller 1340 connecting to the memory 1320 and high-speed expansion ports 1350, and a low speed interface/controller 1360 connecting to a low speed bus 1370 and a storage device 1330. Each of the components 1310, 1320, 1330, 1340, 1350, and 1360, are interconnected using various busses, and may be mounted on a common motherboard or in other manners as appropriate. The processor 1310 can process instructions for execution within the computing device 1300, including instructions stored in the memory 1320 or on the storage device 1330 to display graphical information for a graphical user interface (GUI) on an external input/output device, such as display 1380 coupled to high speed interface 1340. In other implementations, multiple processors and/or multiple buses may be used, as appropriate, along with multiple memories and types of memory. Also, multiple computing devices 1300 may be connected, with each device providing portions of the necessary operations (e.g., as a server bank, a group of blade servers, or a multi-processor system).

The memory 1320 stores information non-transitorily within the computing device 1300. The memory 1320 may be a computer-readable medium, a volatile memory unit(s), or non-volatile memory unit(s). The non-transitory memory 1320 may be physical devices used to store programs (e.g., sequences of instructions) or data (e.g., program state information) on a temporary or permanent basis for use by the computing device 1300. Examples of non-volatile memory include, but are not limited to, flash memory and read-only memory (ROM)/programmable read-only memory (PROM)/erasable programmable read-only memory (EPROM)/electronically erasable programmable read-only memory (EEPROM) (e.g., typically used for firmware, such as boot programs). Examples of volatile memory include, but are not limited to, random access memory (RAM), dynamic random access memory (DRAM), static random access memory (SRAM), phase change memory (PCM) as well as disks or tapes.

The storage device 1330 is capable of providing mass storage for the computing device 1300. In some implementations, the storage device 1330 is a computer-readable medium. In various different implementations, the storage device 1330 may be a floppy disk device, a hard disk device, an optical disk device, or a tape device, a flash memory or other similar solid state memory device, or an array of devices, including devices in a storage area network or other configurations. In additional implementations, a computer program product is tangibly embodied in an information carrier. The computer program product contains instructions that, when executed, perform one or more methods, such as those described above. The information carrier is a computer- or machine-readable medium, such as the memory 1320, the storage device 1330, or memory on processor 1310.

The high speed controller 1340 manages bandwidth-intensive operations for the computing device 1300, while the low speed controller 1360 manages lower bandwidth-intensive operations. Such allocation of duties is exemplary only. In some implementations, the high-speed controller 1340 is coupled to the memory 1320, the display 1380 (e.g., through a graphics processor or accelerator), and to the high-speed expansion ports 1350, which may accept various expansion cards (not shown). In some implementations, the low-speed controller 1360 is coupled to the storage device 1330 and a low-speed expansion port 1390. The low-speed expansion port 1390, which may include various communication ports (e.g., USB, Bluetooth, Ethernet, wireless Ethernet), may be coupled to one or more input/output devices, such as a keyboard, a pointing device, a scanner, or a networking device such as a switch or router, e.g., through a network adapter.

The computing device 1300 may be implemented in a number of different forms, as shown in the figure. For example, it may be implemented as a standard server 1300a or multiple times in a group of such servers 1300a, as a laptop computer 1300b, or as part of a rack server system 1300c.

Various implementations of the systems and techniques described herein can be realized in digital electronic and/or optical circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms “machine-readable medium” and “computer-readable medium” refer to any computer program product, non-transitory computer readable medium, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term “machine-readable signal” refers to any signal used to provide machine instructions and/or data to a programmable processor.

The processes and logic flows described in this specification can be performed by one or more programmable processors, also referred to as data processing hardware, executing one or more computer programs to perform functions by operating on input data and generating output. The processes and logic flows can also be performed by special purpose logic circuitry, e.g., an FPGA (field programmable gate array) or an ASIC (application specific integrated circuit). Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Computer readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

To provide for interaction with a user, one or more aspects of the disclosure can be implemented on a computer having a display device, e.g., a CRT (cathode ray tube), LCD (liquid crystal display) monitor, or touch screen for displaying information to the user and optionally a keyboard and a pointing device, e.g., a mouse or a trackball, by which the user can provide input to the computer. Other kinds of devices can be used to provide interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's client device in response to requests received from the web browser.

A number of implementations have been described. Nevertheless, it will be understood that various modifications may be made without departing from the spirit and scope of the disclosure. Accordingly, other implementations are within the scope of the following claims.

What is claimed is:

1. A computer-implemented method executed on data processing hardware that causes the data processing hardware to perform operations comprising:
receiving a clinical data table for a patient, the clinical data table storing clinical data associated with the patient in tabular form;
extracting, from the clinical data table, one or more categorical features and one or more continuous features;
determining, using a clinical prediction model, one or more predicted clinical outcomes for the patient based on the one or more categorical features and the one or more continuous features extracted from the clinical data table; and
providing, for output from a client device associated with a user, the one or more predicted clinical outcomes for the patient.

2. The computer-implemented method of claim 1, wherein the clinical prediction model executes on the data processing hardware and comprises a clinical tabular multi-head attention model, the clinical tabular multi-head attention model comprising:
a categorical feature encoder configured to:
receive, as input, each categorical feature of the one or more categorical features extracted from the clinical data table; and
generate, as output, a corresponding categorical embeddings for each categorical feature;
a continuous feature encoder configured to:
receive, as input, each continuous feature of the one or more categorical features extracted from the clinical data table; and
generate, as output, a corresponding continuous feature embedding for each categorical feature;
a concatenator configured to concatenate the one or more categorical feature embeddings and the one or more continuous feature embeddings to form a set of parametric embeddings;
a multi-head attention network configured to:
receive, as input, each parametric embedding in the set of parametric embeddings formed by the concatenator; and
generate, as output, a corresponding contextual embedding for each parametric embedding in the set of parametric embeddings; and
a fully-connected feed forward network configured to:
receive, as input, the contextual embeddings generated as output from the multi-head attention network; and
predict, as output, the one or more clinical outcomes for the patient.

3. The computer-implemented method of claim 2, wherein the multi-head attention network comprises a stack of N layers that each comprise a multi-head attention mechanism.

4. The computer-implemented method of claim 2, wherein the multi-head attention network comprises a stack of N Transformer layers.

5. The computer-implemented method of claim 4, wherein each Transformer layer in the stack of N Transformer layers comprises a normalization layer, a masked multi-head attention layer, residual connections, and a feedforward layer.

6. The computer-implemented method of claim 2, wherein:
the one or more clinical outcomes predicted for the patient comprises multiple clinical outcomes predicted for the patient; and
the fully-connected feed forward network comprises multiple heads each configured to:
receive, as input, the contextual embeddings generated as output from the multi-head attention network; and
predict, as output, a respective one of the multiple clinical outcomes for the patient.

7. The computer-implemented method of claim 6, wherein the clinical tabular multi-head attention model is trained via multi-task learning to jointly teach the clinical tabular multi-head attention model to learn how to predict the multiple clinical outcomes for the patient.

8. The computer-implemented method of claim 1, wherein the clinical prediction model executes on the data processing hardware and comprises a large language model.

9. The computer-implemented method of claim 8, wherein the operations further comprise:
serializing the one or more categorical features and the one or more continuous features extracted from the clinical data table into an input text sequence,
wherein determining the one or more predicted clinical outcomes for the patient comprises processing, using the large language model, the input text sequence to generate the one or more predicted clinical outcomes.

10. The computer-implemented method of claim 8, wherein the large language model comprises a pre-trained large language model and is fine-tuned using few-shot learning.

11. The computer-implemented method of claim 8, wherein the large language model comprises a domain-specific large language model pre-trained on a vocabulary and/or syntax associated with particular domain.

12. The computer-implemented method of claim 11, wherein the particular domain comprises medical terminology.

13. The computer-implemented method of claim 1, wherein the one or more predicted clinical outcomes comprise at least one of overall survival, progression-free survival, or a best overall response.

14. The computer-implemented method of claim 1, wherein the one or more predicted clinical outcomes comprises at least one of a recommended treatment or a prognostic biomarker score.

15. A system comprising:
data processing hardware; and
memory hardware in communication with the data processing hardware, the memory hardware storing instructions that when executed on the data processing hardware cause the data processing hardware to perform operations comprising:
receiving a clinical data table for a patient, the clinical data table storing clinical data associated with the patient in tabular form;
extracting, from the clinical data table, one or more categorical features and one or more continuous features;
determining, using a clinical prediction model, one or more predicted clinical outcomes for the patient based on the one or more categorical features and the one or more continuous features extracted from the clinical data table; and
providing, for output from a client device associated with a user, the one or more predicted clinical outcomes for the patient.

16. The system claim 15, wherein the clinical prediction model executes on the data processing hardware and comprises a clinical tabular multi-head attention model, the clinical tabular multi-head attention model comprising:
a categorical feature encoder configured to:
receive, as input, each categorical feature of the one or more categorical features extracted from the clinical data table; and
generate, as output, a corresponding categorical embeddings for each categorical feature;
a continuous feature encoder configured to:
receive, as input, each continuous feature of the one or more categorical features extracted from the clinical data table; and
generate, as output, a corresponding continuous feature embedding for each categorical feature;
a concatenator configured to concatenate the one or more categorical feature embeddings and the one or more continuous feature embeddings to form a set of parametric embeddings;
a multi-head attention network configured to:
receive, as input, each parametric embedding in the set of parametric embeddings formed by the concatenator; and
generate, as output, a corresponding contextual embedding for each parametric embedding in the set of parametric embeddings; and
a fully-connected feed forward network configured to:
receive, as input, the contextual embeddings generated as output from the multi-head attention network; and
predict, as output, the one or more clinical outcomes for the patient.

17. The system claim 16, wherein the multi-head attention network comprises a stack of N layers that each comprise a multi-head attention mechanism.

18. The system claim 16, wherein the multi-head attention network comprises a stack of N Transformer layers.

19. The system claim 18, wherein each Transformer layer in the stack of N Transformer layers comprises a normalization layer, a masked multi-head attention layer, residual connections, and a feedforward layer.

20. The system claim 16, wherein:
the one or more clinical outcomes predicted for the patient comprises multiple clinical outcomes predicted for the patient; and
the fully-connected feed forward network comprises multiple heads each configured to:
receive, as input, the contextual embeddings generated as output from the multi-head attention network; and
predict, as output, a respective one of the multiple clinical outcomes for the patient.

21. The system claim 20, wherein the clinical tabular multi-head attention model is trained via multi-task learning to jointly teach the clinical tabular multi-head attention model to learn how to predict the multiple clinical outcomes for the patient.

22. The system claim 15, wherein the clinical prediction model executes on the data processing hardware and comprises a large language model.

23. The system claim 22, wherein the operations further comprise:
serializing the one or more categorical features and the one or more continuous features extracted from the clinical data table into an input text sequence,
wherein determining the one or more predicted clinical outcomes for the patient comprises processing, using the large language model, the input text sequence to generate the one or more predicted clinical outcomes.

24. The system claim 22, wherein the large language model comprises a pre-trained large language model and is fine-tuned using few-shot learning.

25. The system claim 22, wherein the large language model comprises a domain-specific large language model pre-trained on a vocabulary and/or syntax associated with a particular domain.

26. The system claim 25, wherein the particular domain comprises medical terminology.

27. The system claim 15, wherein the one or more predicted clinical outcomes comprise at least one of overall survival, progression-free survival, or best overall response.

28. The system claim 15, wherein the one or more predicted clinical outcomes comprises at least one of a recommended treatment or a prognostic biomarker score.
